SEQUENCE LISTING (6 sequences)

SEQ ID NO: 1 - protein, 57 aa, Homo sapiens
AQEPVKGPVSTKPGSCPIILIRCAMLNPPNRCLKDTDCPGIKKCCEGSCGMACFVPQ

SEQ ID NO: 2 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 3 - DNA, 623 bp, Homo sapiens
aggccaagctggactgcataaagattggtatggccttagctcttagccaaacaccttcctgacaccatgagggccagcagcttcttgatcgtggtggtgttcctcatcgctgggacgctggttctagaggcagctgtcacgggagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccaaaaaaaaaaaaaaaaaaaaaaaaaaaa

SEQ ID NO: 4 - protein, 15 aa, Artificial
  Tryptic peptide
KCCEGSCGMACFVPQ

SEQ ID NO: 5 - protein, 15 aa, Artificial
  Tryptic peptide
KCCEGSCGMACFVPQ

SEQ ID NO: 6 - protein, 6 aa, Artificial
  Repeat sequence
GQDXVK

(19) United States
(12) Patent Application Publication
(10) Pub. No.: US 2014/0287985 A1
(43) Pub. Date: Sep. 25, 2014

(54) NOVEL USES OF ELAFIN

(71) Applicants: Oliver Wiedow, Kiel (DE); Birge Bargmann, Kiel (DE); Barbara Kahlke, Melsdorf (DE); Lee Shaw, Kiel (DE); Nils Wichmann, Kiel (DE)

(72) Inventors: Oliver Wiedow, Kiel (DE); Birge Bargmann, Kiel (DE); Barbara Kahlke, Melsdorf (DE); Lee Shaw, Kiel (DE); Nils Wichmann, Kiel (DE)

(73) Assignee: Proteo Biotech AG, Kiel (DE)

(21) Appl. No.: 14/299,266

(22) Filed: Jun. 9, 2014

Related U.S. Application Data
(62) Division of application No. 13/582,738, filed on Apr. 17, 2013.
(60) Provisional application No. 61/309,932, filed on Mar. 3, 2010.

(30) Foreign Application Priority Data
Mar. 3, 2010 (GB) 1003559.0

(51) Int. Cl. C07K 14/47

(52) U.S. Cl. CPC C07K 14/47
USPC 514/1.4; 514/21.3

(57) ABSTRACT

The invention relates to novel uses of the polypeptide elafin, and/or homologues, derivatives or fragments thereof having inhibitory activity against leukocyte elastase for the prevention and treatment of medical conditions like SIRS.

Serine proteases are attractive targets for the design of enzyme inhibitors, since they are involved in the etiology of several inflammatory diseases. Within the class of serine proteases; human leucocyte elastase (HLE) and proteinase 3 are among the most destructive enzymes in the body.

Elastase is known to degrade a broad variety of tissue components, such as elastic fibres and other structural proteins of the extracellular matrix. Furthermore elastase promotes inflammation due to its ability to induce the formation and release of proinflammatory mediators (e.g. interleukin 8; see Nakamura et al., J Clin Invest 89: 1478-84 (1992) and transforming growth factor alpha; see Kohri et al., Am J Physiol Lung Cell Mol Physiol 283: L531-40 (2002)). In animal models, elastase has seen shown to be very destructive in organs such as the kidney. Proteinase 3 is less well understood, but acts in principle in a similar manner to elastase.

It also degrades a variety of tissue components and in animal experiments it has been shown to be equally destructive. An important specific action of proteinase 3 is the activation of tumour necrosis factor alpha, which is the most potent inflammatory mediator in man (Coeshott et al., Proc Natl. Acad Sci USA. 96, 6261-8 (1999)).

Human leukocyte elastase and proteinase 3 normally promote healthy tissue degradation preceding remodelling processes. However, due to their degradative activities, these enzymes represent a potential hazard to the integrity of the affected organ. The activity of these and other inflammatory proteases is therefore tightly regulated by a number of specific endogenous protease inhibitor peptides, often referred to collectively as an anti-protease screen.

Despite this protection, the local protease activity can reach levels that overwhelm the anti-protease capacity, leading to considerable tissue damage and the resultant pathologies inflammatory state.

Neutrophil-mediated inflammatory reactions are a common response to tissue damage, invasion by foreign materials and infections by pathogens. Their function is primarily the removal of damaged tissue or the destruction of the invading agents. In localized reactions this type of inflammation is characterized by a massive tissue infiltration with several phagocytic cell types, of which neutrophils are the most numerous and aggressive.

Inflammatory reactions may also occur systemically, leading to a syndrome referred to as systemic inflammatory response syndrome (SIRS). It is closely related to sepsis. SIRS can be triggered by a variety of factors, such as infections, burns, pancreatitis, trauma and following major surgery. Many patients with SIRS may present with varying degrees of organ dysfunction and some may progress to shock with multiple organ dysfunction syndrome (MODS). Inappropriately activated neutrophils sequestered into various tissues play a central role in the pathology of SIRS and MODS [Dallegri F, Ottonello L (1997). Tissue injury in neutrophilic inflammation. Inflamm Res. 46, 382-91].

These cells release significant amounts of elastolytic enzymes which make a considerable contribution to the release of pro-inflammatory mediators as well as the endothelial and tissue injury observed in these conditions. Occurrence of SIRS has been associated with numerous potential causes however no clear picture emerges so far. In a perspective overview by Roger Bone (Immunological Dissonance: continuing evolution in our understanding of the systemic inflammatory response syndrome (SIRS) and the multiple organ dysfunction syndrome (MODS), Annals of Internal Medicine, 15 Oct. 1996, Vol. 125, issue 8, pages 680-687), the various possibilities for an outbreak of either SIRS or MODS are described. The author describes that in the beginning of the 1990's, it was thought that on the basis of an identification of several of the mediators which were thought to cause the reaction underlying sepsis and multiple organ failure, it would be possible to briefly arrive at a solution for an appropriate medication of SIRS and MODS as well as sepsis. Clinical trials were investigating various agents which—as it was then hoped—would downregulate these mediators; however, the author continues to state that the results of these trials were “uniformly disappointing”. Thus, although today multiple organ failure affects as many as 40% of critically ill patients and remains the leading cause of death in intensive care units, it is still thought that multiple organ failure might be an intractable problem and that the underlying inflammatory reaction is vastly more complicated than had been thought earlier.

The degradative activity of neutrophils is largely due to the two serine proteases as mentioned above, i.e. elastase and proteinase-3. In quiescent neutrophils these enzymes are located in cytosolic azurophilic granules. On cell activation by phagocytosis or proinflammatory mediators of bacterial origin such as LPS and N-formyl-methionine peptides; as well as by endogenous factors like TNFα, GM-CSF, complement factor C5a, leukotriene B4 and IL-8, these enzymes are either transferred to phagosomes or exported to the plasma membrane from where they may be released into the extracellular medium. Activated neutrophils are very short-lived and their disintegration leads to the release of significant amounts of these enzymes into the surrounding extracellular space.

Elastase and proteinase-3 play an active role in initiating and sustaining an inflammatory response. They are known to promote the following pro-inflammatory processes:

### Extracellular Matrix and Cellular Protein Degradation

The tissue injury elicited by leukocyte proteases is largely due to their ability to degrade elastin, laminin, fibronectin, collagen and proteoglycans as well as cell surface proteins. The specifying of proteinases-3 for extracellular matrix proteins is similar to, though not identical with that of elastase.

### Cytokine Activation and Induction:

Inflammatory stimuli lead to the acute release of a cascade of proinflammatory cytokines, such as TNFα, IL-1β, IL-6, IL-8 and IL-18, into the tissue fluid and circulation. Elastase and proteinase-3 may up-regulate the synthesis, release and processing of these cytokines:

1. Proteinase-3 activates TNFα

2. Elastase and proteinase-3 participate in the activation of IL-8

3. Proteinase-3 activates IL-1β and IL-18

4. Induction of IL-6 expression

### Activation of Matrix Metalloproteinases

The matrix metalloproteinases (MMP) are a diverse group of related proteases whose signature property is their ability to cleave collagen and other proteins of the extracellular matrix.

MMPs are synthesized as inactive zymogens and require selective proteolysis for activation. Leukocyte elastase is known to process pro-MMP-9 to its active form, while proteinase-3 activates MMP-2. In addition, leukocyte elastase can induce the expression of MMP-2 by macrophages.

MMP-9 in vivo is specifically regulated by a polypeptide inhibitor called TIMP-1 (tissue inhibitor of metallproteases-1). Leuckocyte elastase can degrade TIMP-1 leading to increased unopposed MMP-9 activity.

The successful treatment of inflammatory diseases in man is limited by the availability of drugs with independent mechanisms of action. In particular, no current medicaments are able to suppress the tissue degradation that accompanies many inflammatory processes. The discovery of endogenous antiproteases together with the development of efficient recombinant protein expression systems has raised the hope of emptying these natural body defences pharmaceutically as agents to combat inflammation. These anti-proteases offer considerable potential advantages over currant anti-inflammatory drugs in terms of their potency, specificity and tolerance. Human elafin (the primary structure of which is depicted in SEQ ID NO: 1 of the present application) is one such anti-protease. It is a highly specific, potent and reversible inhibitor for neutrophil-derived elastase and proteinase 3. Although its activity in the body is largely restricted to epithelial tissues, it was also found to be effective in other organs and tissues.

The present invention thus concerns in particular the use of a polypeptide comprising the sequence of SEQ ID NO: 1 or homologues, derivatives or fragments thereof having inhibitory activity against leukocyte elastase for the preparation of a pharmaceutical composition for the prevention and/or treatment of inflammatory diseases; therein, the inflammatory diseases are selected from the group consisting of:
SIRS
MODS
Post-operative inflammatory reactions and
Sepsis, preferably SIRS and MODS.

As discussed in the above description of the prior art, the actual aetiology of SIRS and MODS were unknown; it was however known that several interconnected pathways would probably play a role. Further, several attempts at providing a suitable treatment failed. If is therefore particularly surprising that Elafin had the advantageous effects as shown in the present application and proved to be a suitable active ingredient of a pharmaceutical composition for the prevention and/or treatment of the above-mentioned diseases, in particular SIRS and MODS.

The present inventors could further show that the present polypeptides were particularly useful for the above indications in view of the fact that the inhibition of leukocyte elastase continued for up to 12 hours. This was particularly surprising in view of the fact that the half-life of elafin in plasma is only 115 minutes on average.

Thus, the present polypeptide, homologue, derivative or fragment thereof can be given as a bolus administration before, during or after surgery, without the necessity of repeated administration and still prevent the outbreak of a systemic inflammation response syndrome (SIRS), MODS and in particular post-operative inflammatory reactions, as well as sepsis. A “bolus” administration in the present context shall mean an administration, which is carried out only once or twice, preferably once, to achieve the desired effect as described above. In the context of an intravenous bolus, the administration should preferably have a duration of not more than approximately 60 min. (infusion), preferably not more than 30 min. However, in an alternative embodiment the infusion could be continued for up to 12 h, or op to 24 h.

In view of the fact that the present polypeptide, homologue, derivative or fragment thereof is particularly useful for the preparation of a pharmaceutical composition for the prevention and/or treatment of SIRS, MODS, post-operative inflammatory reactions and sepsis, the present invention contemplates in a further embodiment the use of the above-described compound for the preparation of wounds dressings and/or as an additive to an organ perfusion medium.

In both cases, the elafin compound as defined above and in the claims as enclosed herewith will be useful in the inhibition of leukocyte elastase and thus in the suppression, prevention and treatment of systemic inflammatory response syndrome, MODS, post-operative inflammatory reactions and sepsis.

In a preferred embodiment, the elafin compound as described above is administered intravenously. Particularly preferred, the present Elafin compound can be administered in a dosage of between 10 and 600 mg/day, or in a particular preferred embodiment, as an intravenous bolus of 100 mg to 400 mg, and preferably about 200 mg/day.

In another embodiment, the elafin compound can be administered subcutaneously. In subcutaneous administration, the preferred dosage is between 10 and 800 mg/day, or in a particular preferred embodiment, as a subcutaneous bolus of 50 mg to 400 mg, and preferably about 100 mg/day.

In a further preferred embodiment, administration can be carried out as a preventive administration before, during or shortly after surgery. Preferred is an administration before surgery, even more preferred a bolus (preferably once or twice and more preferred once) subcutaneously.

“Before” surgery, in accordance with the present invention means an administration, which is carried out within 10 hours, preferably 6 hours, even more preferred, 4 hours, further preferred, 2 hours, or most preferred, within 1 min, to one hour before the beginning of surgery, or with the beginning of surgery.

“Shortly after” surgery in the present context means a period of up to 10 hours after the end of surgery; preferably within 8, more preferred, within 2 and even more preferred within one hour after surgery.

Thus, for the first time, the present invention provides a possibility to prevent an outbreak or ameliorate the severity of the above-mentioned diseases, in particular SIRS and MODS, in particular after surgical intervention, by a preferred administration mode, which only necessitates one (or two), preferably intravenous or subcutaneous administrations of Elafin (homologues, derivatives or fragments thereof). This is particularly advantageous in that SIRS and MODS as described above are still one of the leading causes for death of persons after surgery admitted to intensive care units and in that no actual successful treatment has been provided so far, even less any possibility of a successful prevention.

This possibility is particularly surprising in view of the prior art assumptions that in view of the short half-life of elafin in the circulation, it would have to be assumed that a preventive (bolus) administration would not have been possible; the further addition of another medicament to be administered continuously during surgery is however for several reasons disadvantageous, in particular for the reason that it is usually already necessary to continuously administer several further medicaments during surgery, for example antibiotics.

In accordance with the above described embodiments, the present elafin compound can also be used as an advantageous additive for the preparation of medical devices. One example of such a medical device would be an organ perfusion medium, similarly said elafin compound could be used for the preparation of a wound dressing, for the preparation of an additive to an organ perfusion medium, for the preparation of a medical sealant, for the preparation of a coating which is suitable for coating an implant or stent and for the implant or stent per se. In addition, the present invention pertains to an ex vivo method for enhancing the biocompatible of an implant or stent suitable for implantation into the body of a mammal, comprising applying a coating as described above, comprising the elafin compound of the invention, on the implant or stent.

“Elafin Compound” or “Elafin” as used herein above and hereinafter always encompasses the polypeptide comprising the sequence of SEQ ID NO:1 as well as homologues, derivatives or fragments thereof having inhibitory activity against leukocyte elastase.

Elafin was first isolated from the skin of patients with psoriasis, an inflammatory skin disease. It is a soluble protein with 57 amino acids and a molecular weight of about 6 kDa. Cloning of the olefin cDNA revealed that it is synthesized as a 12.3 kDa precursor (117 residues) which is processed intracellularly by cleavage of an N-terminal 22 residue signal sequence to give a 9.9 kDa protein called proelafin (or trappin-2, see below) which is secreted [Molhuizen H O, Alkemade H A, Zeeuwen P L, de Jongh G J, Wieringa B, Schalkwijk J (1993) SKALP/elafin: an elastase inhibitor from cultured human keratinocytes. Purification: cDNA sequence, and evidence for transglutaminase cross-linking. J Biol Chem. 268(16):12028-32; Salienave J M, Silva A (1993) Characterization and gene sequence of the precursor of elafin, an elastase-specific inhibitor in bronchial secretions. Am J Respir Cell Mol Biol. 8, 439-45; Schalkwijk J, Wiedow O, Hirose S (1999) The trappin gene family proteins defined by an N-terminal transglutaminase substrate domain and a C-terminal four-disulphide core. Biochem J. 340, 569-77].

An analysis of the primary structure of proelafin revealed the presence of two functional domains. The N-terminal domain (residues 1-60) contains four repeats of the sequence-Gly-Gln-Asp-X-Val-Lys-(SEQ ID NO:6) which is characteristic of transglutaminase substrates. The glutamine and lysine residues serve as acyl donors and acceptors, respectively, in the transglutaminase-mediated formation of isopeptide inter-protein cross-links [Molhuizen H O, Alkemade H A, Zeeuwen P L, de Jongh G J, Wieringa B, Schalkwijk J (1993) SKALP/elafin; an elastase inhibitor from cultured human keratinocytes. Purification, cDNA sequence, and evidence for transglutaminase cross-linking, J Biol Chem, 268(16):12028-32]. This portion of the molecule is often referred to as the cementoin domain. Tissue transglutaminase is able to cross-link proelafin to a variety of extracellular matrix proteins of the stratum corneum via this domain.

The second domain, consisting of the C-terminal 57 residues, harbours the protease inhibition function of proelafin and is identical to the 6 kDa soluble form of the molecule, i.e. elafin, originally isolated from psoriatic skin. This domain exhibits similarities to members of the whey acidic protein (abbreviated WAP) family in terms of its sequence, protein foldings and arrangement of four characteristic disulphide bridges [Tamechika I, Itakura M, Saruta Y, Furukawa M, Kato A. Tachibana S, Hirose S (1996) Accelerated evolution in inhibitor domains of porcine elafin family members, J Biol Chem. 271, 7012-8; Tsunemi M, Y. Sakakibara S, Katsube Y (1996) Crystal structure of an elastase-specific inhibitor elafin complexes with porcine pancreatic elastase determined at 1.9 Å resolution. Biochemistry 35, 11570-6]. The combination of transglutaminase substrate and WAP domains was subsequently demonstrated in other proteins for which a generic name “the trappin family” was coined. In order to clarify its affiliation with this protein family, the 9.9 kDa proelafin was termed trappin-2. The covalent attachment of proelafin to extracellular matrix proteins has little effect on its ability to inhibit elastase and proteinase-3 [Guyot N, Zani M L, Maurel M C, Dallet-Choisy S, Moreau T (2005) Elafin and its precursor trappin-2 still inhibit neutrophil serine proteinases when they are covalently bound to extracellular matrix proteins by tissue transglutaminase. Biochemistry 44, 15610-8], suggesting that transglutamination is a means of immobilizing this protease inhibitor in an active form.

The mechanism by which elafin is released from proelafin has not been unequivocally elucidated. Proelafin produced in culture by a type II pneumocyte cell line was processed to olefin only in the presence of serum, indicating that cleavage occurs extracellularly, possibly prior to immobilization by transglutaminase [Sallenave J M, Silva A (1993) Characterization and gene sequence of the precursor of elafin, an elastase-specific inhibitor in bronchial secretions. Am J Respir Cell Mol Biol. 8, 439-45]. Consistent with this, tryptase, a mast cell protease was able to selectively release elafin from soluble proelafin [Guyot N, Zani M L, Berger P, Dallet-Choisy S, Moreau T (2005) Proteolytic susceptibility of the serine protease inhibitor trappin-2 (pre-elafin): evidence for tryptase-mediated generation of elafin, Biol Chem. 366, 391-9]. However, tryptase was inactive with proelafin cross-linked to fibronectin [Guyot N, Zani M L. Maurel M C, Dallet-Choisy S, Moreau T (2005) Elafin and its precursor trappin-2 still inhibit neutrophil serine proteinases when they are covalently bound to extracellular matrix proteins by tissue transglutaminase. Biochemistry 44, 15610-8]. Nevertheless, the fact that acid extracts of psoriatic scales only yield elafin and no cementoin domain, suggests that in vivo elafin is cleaved from proelafin cross-linked to skin matrix proteins. Furthermore, analysis of proelafin breakdown products in the urine revealed only the presence of C-terminal sequences, again pointing to the release of elafin by processing of cross-linked proelafin in vivo [Streit V. Wiedow O, Bartels J, Christophers E (1995) Antiprotease activity in urine of patients with inflammatory skin disorders. J Invest Dermatol. 105, 562-6]. The enzymes catalyzing elafin detachment from immobilized proelafin still remain to be identified.

The biosynthesis of proelafin is regulated at the transcriptional level and is strongly enhanced in response to the presence of epithelial inflammatory diseases, such as lymphocytic alveolitis and psoriasis. Physical injury, infections, irritation and exposure to ultraviolet radiation also induce elafin expression in the skin. Accordingly, proinflammatory stimuli such as the cytokines IL-1β and TNFα induce the expression of proelafin and elafin in various cultured cells, including respiratory cells and keratinocytes.

The structure of elafin, methods for its preparation, and its use for treating several disorders have also been addressed in the prior art, in particular in European Patent EP 0 402 068, U.S. Pat. Nos. 5,464,822 and 6,245,739 as well as published US Patent Application 2002/0187635. These references also disclose the primary structure of human elafin as depleted in SEQ ID NO: 1 and its capability to inhibit leukocyte elastase, in particular human leukocyte elastase and porcine pancreatic elastase. Moreover, methods for the preparation of elafin are described in these references. Preparation of derivatives and variants of Elafin is also described in EP 0 662 516 and U.S. Pat. No. 5,734,014. All references above are explicitly incorporated herein for reference in their entirety.

Example 13 shows that Elafin can be administered intravenously to animals such as rats without any side effects. This behaviour was unexpected, since cationic peptides with antimicrobial properties, such as elafin, are frequently haemolytic or cytotoxic.

The invention generally relates to novel uses of polypeptides comprising the sequence depicted in SEQ ID NO: 1 or homologue, derivatives, or fragments of the sequence depicted in SEQ ID NO: 1, for the treatment of medical conditions for which a use of olefin has not yet been contemplated. According to the invention, any polypeptide comprising the sequence of SEQ ID NO: 1 or comprising a homologue, derivative or fragment of the sequence depicted in SEQ ID NO: 1 can be used which exhibits inhibitory activity against leukocyte elastase, preferably against human leukocyte elastase. Experiments for measuring the activity of leukocyte elastase and its inhibition can be conducted by the method described in European Patent EP 0 402 068. As used herein, the term “homologue” refers to peptides or polypeptides which share a substantial degree of homology on the amino acid level with the sequence of SEQ ID NO: 1 over a certain stretch of its primary structure. In particular, the term “homologue” relates to polypeptides having a sequence (or comprising such sequence) which differs from the sequence depicted in SEQ ID NO: 1 by the substitution (or deletion) of one or more single amino acids. Generally, any amino acid from the sequence depicted in SEQ ID NO: 1 can be deleted or substituted against another amino acid as long as the inhibitory activity of the polypeptide is not lost. Further polypeptides are also included which differ from the sequence of SEQ ID NO: 1 by the insertion of one or more additional amino acids. “One or more” in the above context always refers to 1-50, preferably 1-20, even more preferably 1-10, most preferably 1-5.

Based on the amount of identical amino acids, the sequence homology of a homologue according to the invention is usually more than 60%, preferably more than 70%, more than 80%, more than 90%, more than 95%, and even more preferably more than 98% compared to the polypeptide shown in SEQ ID NO: 1. The degree of amino acid homology may be evaluated by use of suitable computer programs known in the art, such as the GCG program package. A degree of homology, which is used throughout this description interchangeably with “identity”, can be determined also by hybridisation techniques, which are well known to a person skilled in the art. The above percentages of identity are thus determined in a preferred embodiment under stringent hybridization conditions.

Identity may be measured using sequence analysis software (e.g., ClustalW at PBIL (Pole Bioinformatique Lyonnaise) http://npsa-pbil.ibcp.fr).

As is known in the art, a number of different programs can be used to identify whether a nucleic acid or amino acid sequence has identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol., Biol. 48,443 (1970), by the search for similarity method of Pearson & Lipman, Proc. Natl. Acad. Sci, USA 85, 2444 (1988), by computerised implementations of these algorithms (GAP, BESTFIT, FASTA, and TFASTA in the Wisconsin Genetics Software Package, Genetics computer Group, 575 Science Drive, Madison, Wis.), the Best Fit sequence program described by Devereux et al., Nucl. Acid Res. 12, 387-395 (1984), preferably using the default settings, or by inspection.

The present invention also provides the inventive polypeptides expressed recombinantly in a suitable host from a corresponding polynucleotide. The present invention particularly provides such polynucleotides, which hybridize under stringent conditions to corresponding polynucleotides. As herein used, the term “stringent conditions” means conditions which permit hybridization between polynucleotides sequences and the polynucleotide sequences of SEQ ID NO: 1 where there is at least about 70% identity.

Suitably stringent conditions can be defined by, e. g., the concentrations of salt or formamide in the prehybridization and hybridization solutions, or by the hybridization temperature, and are well known in the art. In particular stringency can be increased by reducing the concentration of salt, by increasing the concentration of formamide, and/or by raising the hybridization temperature.

For example, hybridization under high stringency conditions may employ about 50% formamide at about 37° C. to 42° C., whereas hybridisation under reduced stringency conditions might employ about 35% to 25% formamide at about 30° C. to 35° C. One particular set of conditions for hybridization under high stringency conditions employs 42° C., 50% formamide, 5×. SSPE, 0.3% SDS, and 200 pg/ml sheared and denatured salmon sperm DNA. For hybridization under reduced stringency, similar conditions as described above may be used in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

Thus, the term homologue also comprises polypeptides which are longer than the sequence of SEQ ID NO: 1 and therefore comprise more amino acids. Insofar as a part of their amino acid sequence shares substantial homology with the polypeptide of SEQ ID NO: 1. A polypeptide comprising the sequence depleted in SEQ ID NO: 1 which can be used according to the invention is, for example, the preproelafin shown in SEQ ID NO: 2 (Moihuizen, H. O. et al., J. Biol. Chem. 268 (16), 12028-12032 (1993)). As can be seen from that sequence, preproelafin comprises an additional N-terminal extension on the N-terminus and is post-translationally cleaved to provide the mature form. Preproelafin (117 amino acids) is first cleaved in proelafin (95 amino acids) and the N-terminal signal peptide (22 amino acids). During the terminal differentiation of keratinocytes (formation of the horny layer), proelafin becomes crosslinked to comified envelope proteins by epidermal transglutaminase. The mature elafin is apparently released from these comified envelope proteins by a yet unknown mechanism and can be extracted from horny layers of human skin, particularly from scales of patients suffering from psoriasis.

According to the present invention, the term “fragment” refers to any biologically active portion of the polypeptide in SEQ ID NO: 1 having the desired enzymatic activity. A fragment of elafin can consist of an amino acid sequence differing from the amino acid sequence in SEQ ID NO: 1 by the deletion of one or more amino acids at the N-terminus and/or C-terminus. For example, a fragment according to the invention may lack amino acid residue(s) 1, 1-2, 1-3, 1-4, 1-5, 1-6, 1-10 or 1-20 at the N-terminus of the polypeptide. Similarly, it may lack the corresponding residues at the C-terminus.

Moreover, a fragment may differ from the amino acid sequence in SEQ ID NO: 1 by lacking amino acid residues at both the N-terminus and C-terminus. For example, a fragment may consist of amino acids 6-30 of the polypeptide shown in SEQ ID NO: 1. Further comprised by the invention is the use of homologues of the fragments of the polypeptide shown in SEQ ID NO: 1.

The term “derivatives” refers to peptides or polypeptides which differ from the polypeptide shown in SEQ ID NO: 1 or from the homologs and fragments derived therefrom by amino acid modifications, such as glycosylate, PEGylation, biotinylation, cyclization and/or oxidation [see Example 14]. It is preferred that the fragments, homologues and derivatives of the invention inhibit human leukocyte elastase at least 50%, more preferably 75%, 80%, 80%, 85% or 99% as compared to the polypeptide of SEQ ID NO:1. Expressed in terms of Ki, the fragments, homologues and derivatives of the invention show values of about 10⁻⁶ M to 10⁻¹⁴ M, preferably 10⁻⁸ M to 10⁻¹² M, and more preferably 10⁻⁹ M to 10⁻¹¹ M, whereas elafin exhibits a Ki of about 10⁻¹⁰ M.

The polypeptides of the invention can be obtained as described in the prior art (see, for example, EP 0 402 068) or can be prepared by recombinant expression of the coding sequence as described by Salienave, J. M. and Silva, A (Am. J. Respir. Cell Mol. Biol. 8 (4), 439-445 (1993)). The coding sequence is also provided in SEQ ID NO: 3. The polypeptides, homologues, derivatives and fragments as defined herein may be used as obtained or purified in a known and appropriate manner and formulated into pharmaceutical compositions, for example by admixture with a pharmaceutically acceptable diluent or carrier. Administration may be by way of various routes known in the art. In particular, administration may be effected parenterally, for example intra-nasally, intravenously, rectally, pulmonary, and by way of injection such as by way of intramuscular or subcutaneous injection. The pharmaceutical compositions will be formulated according to the mode of administration to be employed. For example, when the composition is to be administered intra-nasally, the composition may be formulated as a powdered aerosol; and when the composition is to be administered by way of injection it may be formulated as a sterile solution or suspension.

Suitable diluents including aqueous solutions and additives, such as buffers and surfactants may be added.

Pharmaceutical compositions of the present invention also include controlled release formulations. For example, the polypeptides of the present invention may be encapsulated in a biodegradable polymer, or may be dispersed in a matrix of such a polymer, so that the polypeptide is released as the degradation of the polymer matrix proceeds.

Suitable biodegradable polymers for use in sustained release foundations include polyesters which gradually become degraded by hydrolysis when placed in an aqueous, physiological environment. A particular pharmaceutical composition which provides for extended release of a poly-peptide is described in European Patent No. 0058481. In this composition a polylactide is employed, and when placed in an aqueous physiological-type environment, the polypeptide is released from the composition in a continuous manner until essentially all of the polypeptide has been released. Such polymers therefore offer the advantage of a highly localised target area, thus minimizing dosage and any potential side effects.

In the present application, reference to “Elafin” shall encompass “homologues, derivatives and fragments” as described above.

Elafin may be administered systemically or by use of microspheres incorporated e.g. into a medical device, e.g. into a stent or implant which release the elafin in a controlled manner. The elafin can also be used in the form of elafin-containing polymers that release elafin in a controlled manner [Example 15]. The high stability of elafin to ethylene oxide sterilization [Example 12] is a further important aspect for its therapeutic application in this context, especially if is to remain active as a slow release drug or as a component in a medical device coating. The biocompatibility of intravenously administered elafin has been proven. [Example 13]

Furthermore, the invention relates to the use of a polypeptide comprising the sequence of SEQ ID NO: 1 or homologues, derivatives, or fragments thereof having inhibitory activity against leukocyte elastase for coating implants or stents. The invention also pertains to implant or stents coatings comprising a polypeptide comprising the sequence of SEQ ID NO:1 or homologues, derivatives, or fragments thereof having inhibitory activity against leukocyte elastase and implants and stents comprising such coatings.

The unspecific immunological stimulation of leukocytes after contact with synthetic surfaces often causes an inflammatory response, in severe cases an outbreak of SIRS or MODS. Here, it was found that the application of elafin is effective in overcoming these problems. Elafin has turned out to be useful for coating implants or stents to enhance their biocompatible. It is preferred to use elafin in long term implantable devices such as natural or synthetic vascular grafts, natural or synthetic heart valves, indwelling catheters for dialysis, dental and orthopedic implants, artificial joints, materials for osteosynthesis, probes of cardiac pacemakers or long-term perfusion. In this manner, rejection of implants can be avoided or attenuated, by preventing a possible outbreak of SIRS/MODS.

According to a further aspect, the invention relates to the use of a polypeptide comprising the sequence of SEQ ID NO: 1 or homologues, derivatives, or fragments thereof having inhibitory activity against leukocyte elastase in wound dressings. Further, a wound dressing comprising a polypeptide comprising the sequence of SEQ ID NO: 1 or homologues, derivatives, or fragments thereof having inhibitory activity against leukocyte elastase is provided.

Additional diseases such as chronic venous insufficiency or diabetes mellitus frequently impair wound healing. As a consequence wound healing is delayed and local infections as well as inflammation, in severe cases SIRS/MODS, take place. Typical current therapies focus on antimicrobial agents and treatment of the underlying diseases like vascular surgery and diabetes treatment.

The use of conventional wound dressings is limited by the fact that they have no antimicrobial and no anti-inflammatory activities. Thus, they are frequently withdrawn from therapy, it has been found that elafin can be effectively used in wound dressings and upon contact with the wound, promote healing and reduce the risk of infections.

Due to its extraordinary stability, high specificity as well as the human origin, elafin is an ideal active compound to be immobilised on wound pads for chronic wounds or to be applied as a solution or a gel directly to the wound.

According to a further aspect, the invention relates to the use of a polypeptide comprising the sequence of SEQ ID NO: 1 or homologues, derivatives, or fragments thereof having inhibitory activity against leukocyte elastase as an additive of a medical sealant. Preferably the medical sealant is a fibrin-sealant or a bone-cement. The invention also relates to a medical sealant comprising a polypeptide comprising the sequence of SEQ ID NO: 1 or homologues, derivatives, or fragments thereof having inhibitory activity against leukocyte elastase.

Fibrin glue is composed of two separate solutions of fibrinogen and thrombin. When mixed together, these two solutions mimic the final stages of the clotting cascade to form a fibrin clot. Fibrin glue has been used in a wide variety of surgical procedures to repair, seal, and attach tissues in a variety of anatomic sites. The advantage of fibrin glue over other adhesives, such as the cyanoacrylates, is that a natural biomaterial that is completely reabsorbed in 2 weeks to 4 weeks.

Problems can appear because the resulting fibrin patch is a good medium for microbial growth or if the fibrin glue is used to seal inflamed tissue. Innate elastase inhibitors are known to be putatively involved in the regulation of tissue inflammation by inhibiting polymorphonuclear leukocyte (PMN) derived proteinases like elastase.

It was now found that the addition of Elafin promotes healing and prevents loosening of implants because of the inhibition of posttraumatic inflammation. It can be added to any commercially available sealants or bone-cements known in the art [see Example 15].

Elafin appears to be an ideal compound for medical sealants, since, in addition to its anti-protease activity, it has been shown to kill Pseudomanae aeruginosa and Staphylococcus aureus, which are the most important bacteria in wound infections.

### BRIEF DESCRIPTION OF THE FIGURES

The present application comprises the following figures, which are described here below as follows:

FIG. 1: shows the plasma level of Elafin (n=3) at various times during esophagectomy.

FIG. 2: shows the time line of serum elastase activity upon administration of Elafin or placebo.

FIG. 3: shows IL-6 increase in serum two hours postoperative.

FIG. 4: shows IL-8 increase in serum six hours postoperative.

FIG. 5: shows CRP increase postoperative.

FIG. 6: shows the decrease of the necessity for treatment in intensive care.

FIG. 7: shows the duration of serum elastase inhibition after intravenous administration of Elafin.

FIG. 8: shows the elimination of various doses of Elafin from the blood of human volunteers.

FIG. 9: time course of plasma Elafin concentrations after subcutaneous administration

FIG. 10: time course of inhibition of serum elastase after subcutaneous Elafin administration.

### EXAMPLES

### Example 1

Patients undergoing esophagectomy for esophagus carcinoma were administered either 200 mg Elafin in 250 mL physiological saline (verum) or 250 mL saline (placebo) i.v. over a period of 30 min. beginning 15 min. before the commencement of surgery (time point dl −15 min.). Blood samples were removed at the intervals indicated and plasma was prepared and stored frozen at −20° C. Plasma Elafin levels were measured using an ELISA.

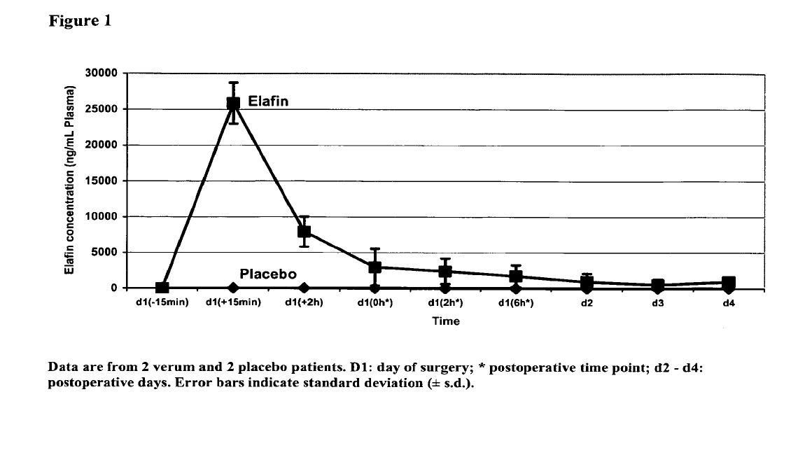

Patients receiving verum treatment exhibited significant plasma Elafin concentrations during surgery and up to 6 h after the end of the operation. Patients receiving placebo treatment had no detectable Elafin in their plasma (see FIG. 1).

### Example 2

Patients undergoing esophagectomy for esophagus carcinoma were administered either 200 mg Elafin in 250 mL physiological saline (verum) or 250 mL saline (placebo) i.v. over a period of 30 min. beginning 15 min. before the commencement of surgery (time point dl −15 min.). Blood samples were removed at the intervals indicated and serum was prepared and stored frozen at −20° C. Serum elastase activity was determined using a photometric assay.

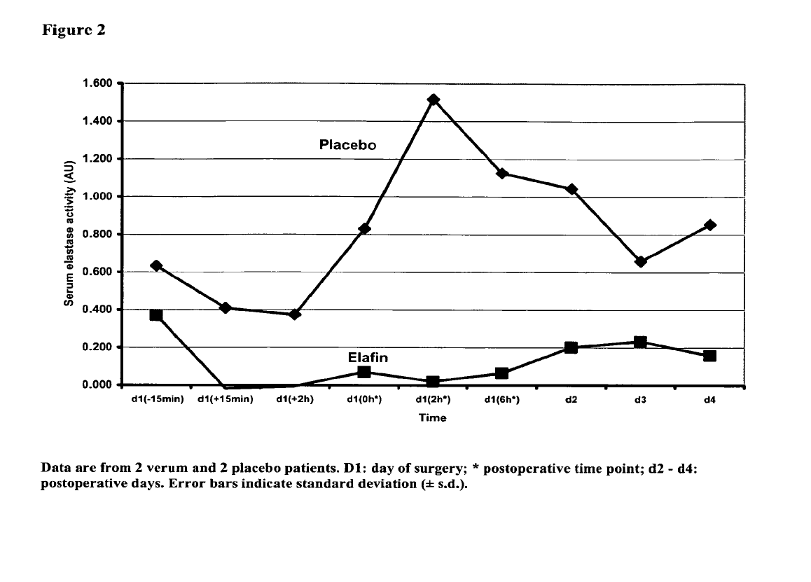

In patients receiving placebo, serum elastase activity was markedly increased during surgery and recovered to a near preoperative level during the subsequent 3 postoperative days. In patients receiving Elafin, serum elastase activity was markedly reduced during the period of surgery and recovered to slightly less than the preoperative level in the course of the subsequent 3 postoperative days (see FIG. 2). The period and extent of elastase inhibition correlate with the plasma levels of Elafin indicated in FIG. 1.

### Example 1

Patients undergoing esophagectomy for esophagus carcinoma were administered either 200 mg Elafin in 250 mL physiological saline (verum) or 250 mL saline (placebo) i.v. over a period of 30 min beginning 15 min before the commencement of surgery. Blood samples were taken 2 h after finishing surgery and serum was prepared. The serum level of IL-6 was measured using a routine immunoassay (“Immulite, IL-6”, available from Siemens Medical Solution Diagnostics).

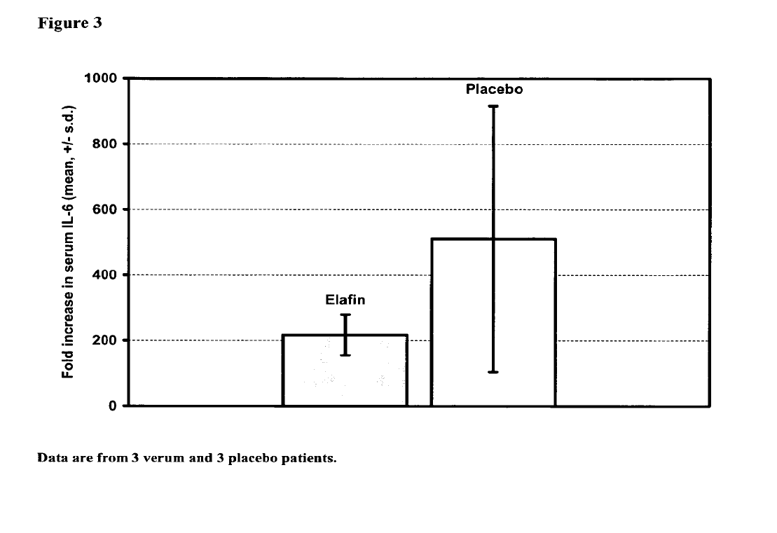

Patients receiving placebo exhibited a marked increase in serum IL-6 2 h postoperatively. The administration of Elafin diminished the postoperative increase in IL-6 by about 80%. As IL-6 is a major inflammatory cytokine, the results demonstrate the significant anti-inflammatory effects of Elafin (see FIG. 3).

### Example 4

Patients undergoing esophagectomy for esophagus carcinoma were administered either 200 mg Elafin in 250 mL physiological saline (verum) or 250 ml saline (placebo) i.v. over a period of 30 min beginning 15 min before the commencement of surgery. Blood samples were taken 6 h after finishing surgery and serum was prepared. The serum level of IL-8 was measured using a commercially available ELISA, (“Immulite, IL-8”, available from Siemens Medical Solution Diagnostics).

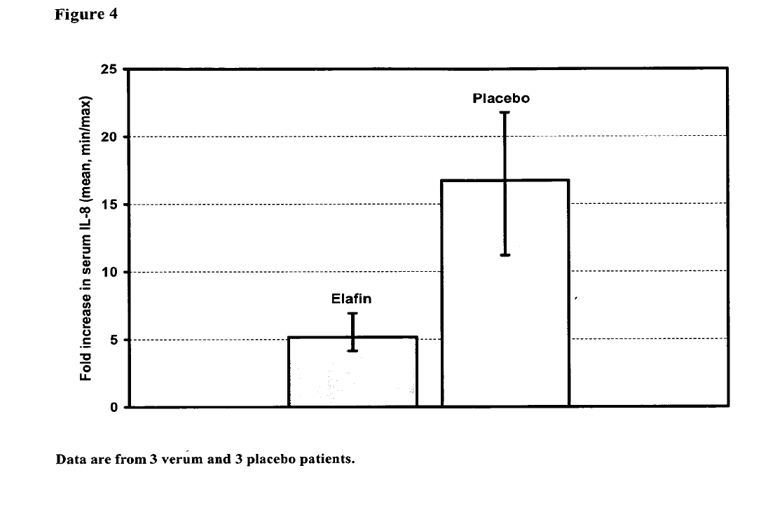

Patients receiving placebo exhibited a marked increase in serum IL-8 6 h postoperatively. The administration of Elafin diminished the postoperative increase in IL-8 by about 70%. As IL-8 is a major inflammatory cytokine/chemokine, the results demonstrate the significant anti-inflammatory effects of Elafin (see FIG. 4).

### Example 5

Patients undergoing esophagectomy for esophagus carcinoma were administered either 200 mg Elafin in 250 mL physiological saline (verum) or 250 mL saline (placebo) i.v. over a period of 30 min beginning 15 min before the commencement of surgery. Serum was prepared from blood samples which were taken on admission and on the day after surgery. The serum levels of CRP were measured using a commercially available ELISA. (CRPL 3, cobas®, available from Roche/Hitachi).

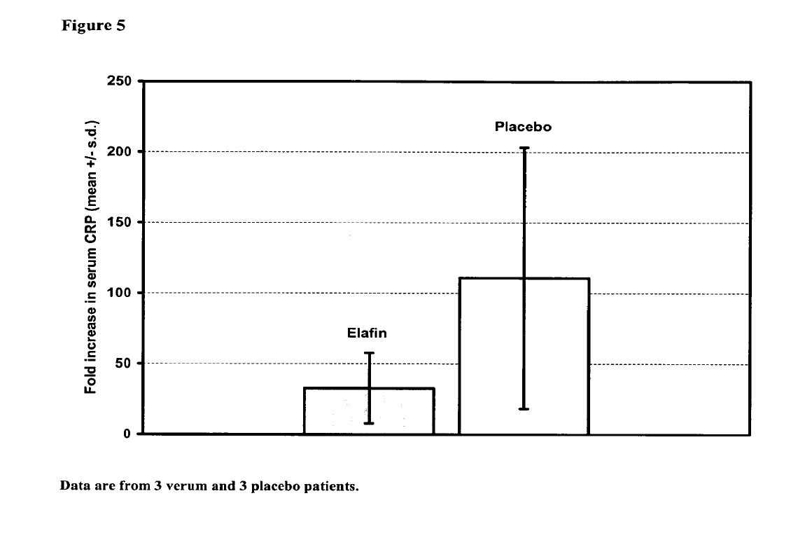

Patients receiving placebo exhibited a marked increase in serum CRP on the first postoperative day. The administration of Elafin diminished this postoperative increase in CRP by about 70% (see FIG. 5). As CRP is a sensitive indicator of inflammation, the results demonstrate the significant anti-inflammatory effects of Elafin.

### Example 6

Patients undergoing esophagectomy for esophagus carcinoma were administered either 200 mg Elafin in 250 mL physiological saline (verum) or 250 mL saline (placebo) i.v. over a period of 30 min beginning 15 min before the commencement of surgery. After surgery all patients were routinely admitted to the intensive care unit (ICU) at least overnight. Patients were maintained in the ICU for as long as the treating physician considered necessary.

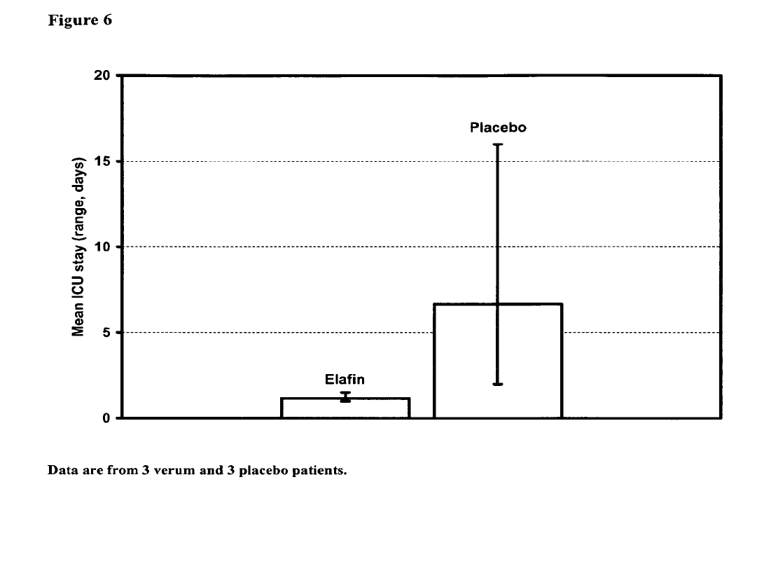

Compared with patients receiving placebo the administration of Elafin led to a significant reduction in the period of postoperative ICU care required (see FIG. 6).

“Significant reduction” in the present context refers to a necessity for intensive care which was reduced by at least 25% or one day.

### Example 7

In order to assess the potential pharmacological effect of administered Elafin the inhibition of leukocyte elastase activity in the serum was measured.

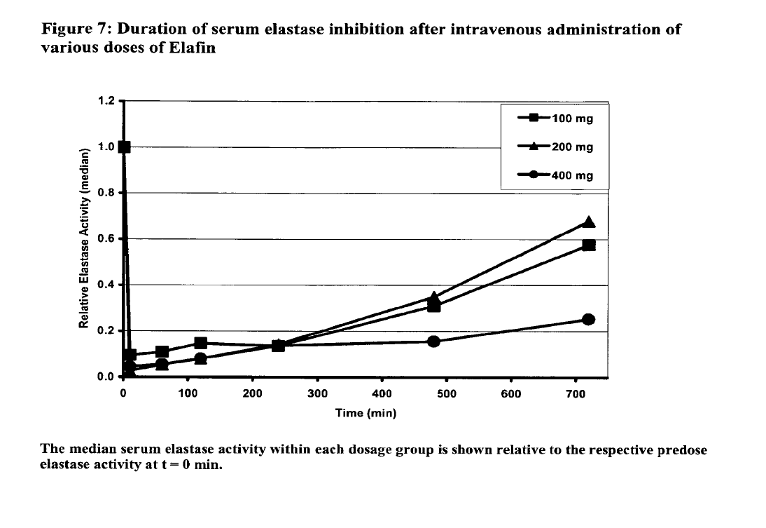

The data presented in FIG. 7 were obtained in a Phase I clinical trial. The data show the time course of serum elastase activity in 6 healthy male volunteers after a single intravenous infusion of various doses of Elafin in 250 mL physiological saline over a period of 30 min.

Within each dosage group the median elastase activity is shown relative to the predose value at t=0 min.

The results show that intravenously administered Elafin causes a rapid and potent inhibition of serum elastase. The recovery of elastase activity is gradual. Assuming an inhibition threshold of 50%, the inhibitory effect of a 100 mg dose of intravenously administered Elafin could be detected up to a period of 720 min after administration (see also Table 2).

### Example 8

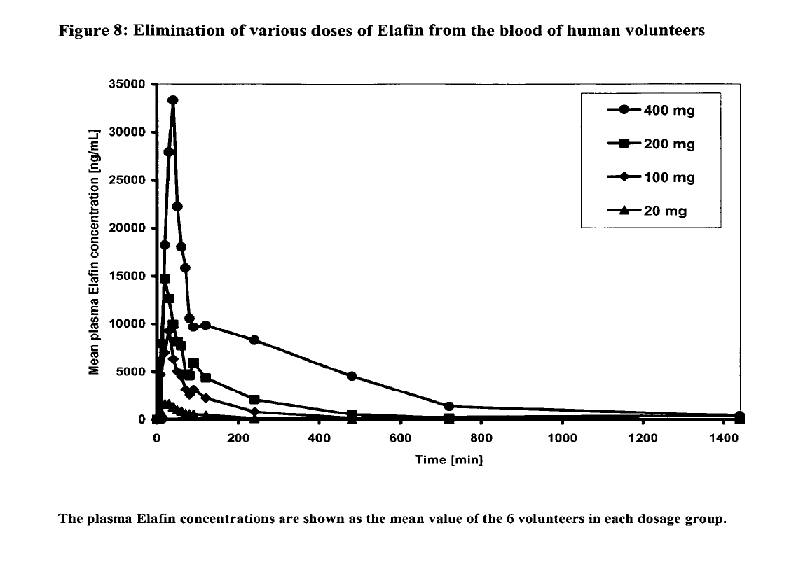

The data presented in FIG. 8 were obtained in a Phase I clinical trial. The data show the time course of Elafin concentrations in healthy male volunteers after a single intravenous infusion of Elafin in 250 mL physiological saline over a period of 30 min. Each dose was administered to 6 individuals. After Elafin administration blood samples were taken at the indicated times from each volunteer and the plasma prepared and frozen to −20° C. Elafin in the plasma samples was quantified using an ELISA.

The presentation of the data show that Elafin is eliminated in two first-order processes, an initial phase of rapid elimination followed by a phase in which Elafin was eliminated more slowly (see FIG. 8). The respective half lives of these two elimination phases (termed t½α and t½β, respectively are presented in Table 1.

TABLE 1
Half life of Elafin elimination from healthy individuals
after intravenous administration

| | Elafin dose | | | |
|---|---|---|---|---|
| | 20 mg | 100 mg | 200 mg | 400 mg |
| t_{1/2}α (min) | 8.99 | 9.65 | 6.48 | 9.82 |
| t_{1/2}β (min) | 75.84 | 88.53 | 115.47 | 135.81 |

### Duration of Inhibition of Serum Elastase by Intravenously Administered Elafin

The time course of inhibition of serum elastase by various doses of Elafin is shown in FIG. 7. From these data, the duration of elastase inhibition was calculated, as judged by the time taken for 50% of elastase activity to recover

TABLE 2
Duration of inhibition of serum elastase by
various intravenous doses of Elafin

| Dose | 20 mg | 100 mg | 200 mg | 400 mg |
|---|---|---|---|---|
| Median t_{50%} [min] | 40 | 720 | 480 | 720 |
| (range) | (10; 90) | (480; 720) | (120; 720) | (480; 720) |

t_{50%} Duration of serum elastase inhibition to a threshold level of 50%.

The results show that Elafin doses of 100-400 mg are able to exert a pharmacologically relevant elastase over period of up to 720 min. (12 hours). Thus, despite the rather rapid elimination of Elafin from the plasma (FIG. 7; Table 1), its pharmacological effects persist for significantly longer.

This conclusion, which is based on data from a Phase I clinical trial on healthy individuals, is confirmed by data acquired from a clinical trial on patients undergoing esophagectomy for esophagus carcinoma (FIGS. 1 and 2). These data show that the elimination of Elafin occurs mainly within the first 2 hours after its intravenous administration at the beginning of surgery (FIG. 1) from which a half life of at most 2 h (120 min) can be estimated.

This value is in agreement with the data from the Phase I trial (FIG. 8 and Table 1).

Furthermore, the duration of elastase inhibition in these patients is longer than might be expected on the basis of its elimination rate (FIG. 2).

### Example 9

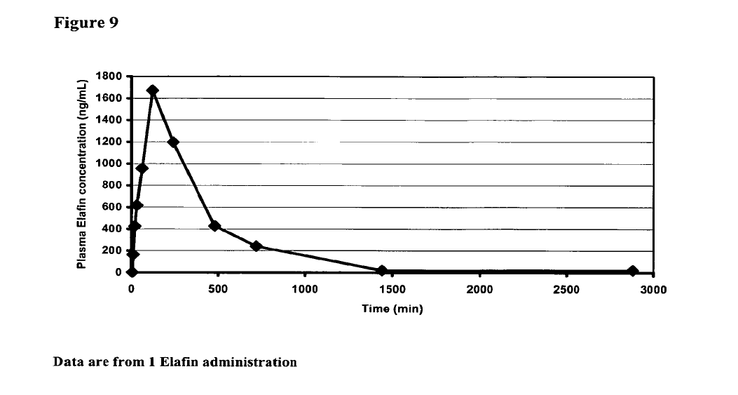

100 mg of Elafin physiological saline were injected subcutaneously into the abdomen of a healthy human volunteer. Blood samples were removed at the times indicated in FIG. 9 and plasma prepared and stored at −20° C. The plasma Elafin levels were measured using an ELISA.

The results show a slow increase in the plasma concentration of Elafin, peaking at about 60 min after administration.

From these data a plasma half life for Elafin of approximately 230 min was estimated (see FIG. 9).

### Example 10

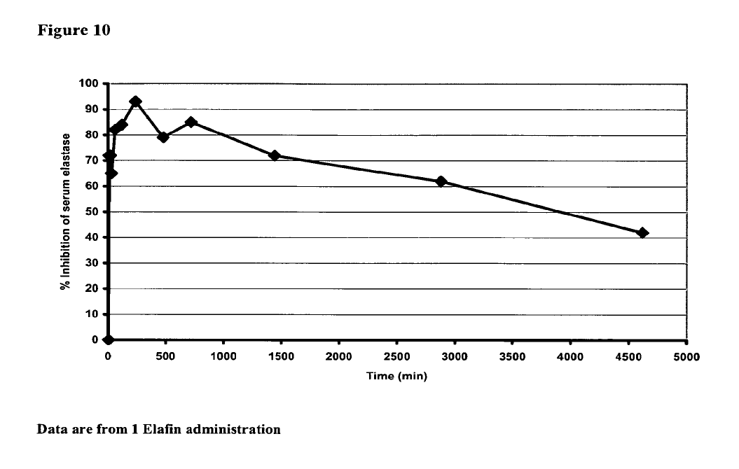

100 mg of Elafin in physiological saline were injected subcutaneously into the abdomen of a healthy human volunteer. Blood samples were removed at the times indicated in FIG. 10 and serum prepared and stored at −20° C.

The results in FIG. 10 show that subcutaneously injected elafin caused a potent inhibition of serum elastase activity within the first 30 min-4 hours after infection. This was followed by a slow recovery of activity.

Assuming an inhibition threshold of 50%, the inhibitory effect of Elafin injected subcutaneously could be detected up to 4000 min (66 hours) after administration (see FIG. 10).

### Summary of Data Relating to Intravenously and Subcutaneously Administered Elafin

The data presented in FIGS. 7-10, Table 1 and Table 2 is summarized in Table 3.

TABLE 3
Comparison of rate of elimination and duration of pharmacological effects of a 100 mg
dose of Elafin administered intravenously or subcutaneously

| | Intravenous administration (n = 6) | Subcutaneous adninistation (n = 1) |
|---|---|---|
| Half life of Elafin elimination | 2 phases with half life 9.65 min and | Estimated half life approx. |
| from plasma | 88.53 min | 230 min |
| Duration of Elafin effects (period | 720 min | 4000 min |
| of at least 50% inhibition | | |
| of serum elastase) | | |

### CONCLUSION

Subcutaneously administered Elafin is eliminated more slowly that intravenous Elafin and exerts its pharmacological effects over a longer period of time.

### Example 12

### Stability of Elafin to Sterilization

| Sample | HPLC | Inhibitory activity |
|---|---|---|
| Control (room temperature) | 108 pg | 100% |
| Heat sterilization | 0 pg | −19- |
| Ethylene oxide sterilization | 100 pg | 101 |

Lyophilized samples of 300 μg Elafin were sterilized either by heat sterilization or by ethylene oxide sterilization in a suitable standard sterile packing material. After sterilization, samples were dissolved in 300 μl distilled water. Elafin was quantified by HPLC (reverse phase C8-separation medium) and by determination of elastase inhibitory activity.

### Example 13

### Biocompatibility of Elafin

Rats were treated with up to 100 mg elafin/kg/day over a period of 14 days by continuous intravenous infusion. No mortality occurred during the course of the study and no clinical signs of haemolysis or cytotoxicity were observed in the skin, eyes, mucous membranes, lungs and circulation. Neither food consumption nor body weight was influenced by the treatment.

### Example 14

### Inhibitory Activity of Elafin Derivatives Towards Elastase

Recombinant elafin produced in a commercial Hansenula expression system was purified by cation exchange chromatography and reverse phase HPLC. The latter chromatographic step yielded several distinct peaks exhibiting elastase inhibitory activity. These were linearized, subjected to trypsin digestion and analysed by mass spectrometry. In one of the HPLC peaks a tryptic peptide with a molecular mass of 1805.67, which is consistent with a fragment KBBEGSBGXABFVPQ (SEQ ID No: 4) (where X represents an oxidized methionine and B is carboxymethyl-cysteine), was identified. The corresponding unmodified Elafin fragment KBBEGSBGMABFVPQ (SEQ ID NO:5) from an unoxidized trypsin-digested sample exhibited a molecular mass of 1789.67. Oxidation of the elastase inhibitor derivative was confirmed by MS/MS sequencing.

### Example 15

To estimate the applicability of a protease inhibitor as a biologically active component of bone cement, elafin was added to the solid component of customary commercial bone cement, consisting of a bead polymer of methyl methacrylate-methyl acrylate copolymer and the polymerization catalyst dibenxoyl peroxide.

After the polymerisation reaction, pieces of the cement were incubated in water for various periods of time. The supernatants were examined for the release of elastase inhibitory activity. Active olefin was released from the polymerized cement samples. Within the first three days, about 15% of the added olefin was released.

1-11. (canceled)

12. A method for the prevention of one or more inflammatory diseases, comprising administering the polypeptide comprising the sequence of SEQ ID NO: 1 or homologues, derivatives, or fragments thereof having inhibitory activity against leukocyte elastase to a subject, wherein homologue is defined as a sequence having at least 60% sequence homology to SEQ ID NO:1, wherein derivative is defined as a sequence having modifications selected from the group of glycosylation, pegylation, biotinylation, cyclization and oxidation, and wherein fragment is defined as a sequence which differs from SEQ ID NO:1 by lacking 1-10 amino acids at the N- or C-terminals.

13. The method according to claim 12, wherein the one or more inflammatory diseases is systemic inflammatory response syndrome (SIRS) and/or multiple-organ dysfunction syndrome (MODS).

14. The method according to claim 12, wherein the polypeptide is administered intravenously.

15. The method according to claim 14, wherein the dosage is between 10 and 600 mg per day and/or per unit-dosage.

16. The method according to claim 14, wherein the administration is an intravenous bolus of 100 mg to 400 mg.

17. The method according to claim 12, wherein the polypeptide is administered subcutaneously.

18. The method according to claim 17, wherein the dosage is between 10 and 600 mg per day and/or per unit-dosage.

19. The method according to claim 12, wherein the administering is carried out before, during or shortly after surgery.

20. The method according to claim 12, wherein the polypeptide is administered not more than twice.
